(19) United States
(12) Patent Application Publication
(10) Pub. No.: US 2025/0318999 A1
(43) Pub. Date: Oct. 16, 2025

(54) Customizable Cosmetic Face Sheet

(71) Applicants: Mimi Rhyann Tafel, Dallas, TX (US); Lara Briggs Tafel, Dallas, TX (US)

(72) Inventors: Mimi Rhyann Tafel, Dallas, TX (US); Lara Briggs Tafel, Dallas, TX (US)

(21) Appl. No.: 18/631,658

(22) Filed: Apr. 10, 2024

(51) Int. Cl. A61K 8/02

(52) U.S. Cl. CPC A61K 8/0212; A61K 2800/87

(57) ABSTRACT

In one example, an apparatus includes a face sheet having a facial shape and an inner surface removably attachable to facial skin. A plurality of biocompatible cosmetics are on the inner surface. Each cosmetic is transferable to facial skin responsive to compressing the face sheet against a human face. A biocompatible adhesive is on the inner surface. The biocompatible adhesive is configured to removably attach the face sheet to facial skin and to remain attached to the face sheet responsive to detachment of the face sheet from facial skin.

### BACKGROUND

The art of applying cosmetics to the face can be a time-consuming endeavor, often requiring one to spend time in front of the mirror to carefully achieve the desired appearance. As this process often demands one's full attention, it can be a challenge to handle other responsibilities effectively. A solution is needed that allows a person to apply personalized cosmetics efficiently and accurately while also enabling that person to simultaneously multitask.

### SUMMARY

In one example, an apparatus includes a face sheet having a facial shape and an inner surface removably attachable to facial skin. A plurality of biocompatible cosmetics are on the inner surface. Each cosmetic is transferable to facial skin responsive to compressing the face sheet against a human face. A biocompatible adhesive is on the inner surface. The biocompatible adhesive is configured to removably attach the face sheet to facial skin and to remain attached to the face sheet responsive to detachment of the face sheet from facial skin.

In another example, a method includes accessing first data corresponding to a three-dimensional image of a user's face, accessing second data corresponding to a customized selection of a plurality of cosmetics, and accessing third data corresponding to a customized selection of a respective facial surface area for each of the plurality of cosmetics. Based on the first, second, and third data, a first cosmetic of the plurality of user-selected cosmetics is removably attached to a first inner surface area of a face sheet. The first inner surface area corresponds to the user's customized selection of the respective facial surface area. The first cosmetic is configured to attach to human skin responsive to pressure applied to an outer surface of the face sheet. Skin-friendly adhesive is applied on the inner surface of the face sheet. The skin-friendly adhesive is configured to removably adhere the face sheet to human skin.

In another example, an apparatus includes a planar face sheet, a plurality of biocompatible cosmetics, and a mask. The planar face sheet has an inner surface and an outer surface. The plurality of biocompatible cosmetics are on the inner surface. The mask is customized in three mutually-orthogonal dimensions to fit a human face. Adhesive is on the outer surface of the planar face sheet. The adhesive is configured to removably attach the face sheet to the mask and to hold the face sheet in place within the mask. Each biocompatible cosmetic is configured to transfer to facial skin, and thereby alter an appearance of facial skin, responsive to a force manually applied to an outer surface of the mask.

### BRIEF DESCRIPTION OF THE DRAWINGS

FIG. 1 is a top-down view of an example inward, skin-facing surface of a face sheet having transferable cosmetics thereon.

FIG. 2 is a top-down view of an example inward, skin-facing surface of an alternative face sheet having transferable cosmetics thereon.

FIG. 3 is a side-view of a wearer preparing to manually apply the face sheet of FIG. 1 to her face, with the inner surface of the face sheet, which has cosmetics thereon, shown as facing the wearer's face.

FIGS. 4A-4C show various perspective views of a portion of an example mask, in which FIG. 4A is a front view, FIG. 4B is a side-view, and FIG. 4C is a back view.

FIG. 5 is a top-down view of the outer surface of the face sheet of FIG. 1.

FIG. 6 is a perspective view of a positioning system that can be used to correctly position the face mask of FIG. 1 on a wearer's face.

FIG. 7 is an exploded side-view showing the orientation of a face-sheet positioning system relative to a wearer's face, which may be used to assist in accurately positioning the face sheet of FIG. 1 on the wearer's face and to accurately transfer cosmetics to facial skin.

FIG. 8 is a side-view of the face-sheet positioning system of FIG. 7, in which a wearer has manipulated the positioning system against her face and is in the process of manually compressing the positioning system (and hence its adhered face mask and the cosmetics thereon) against her face, thereby initiating transfer of cosmetics to her facial skin.

FIG. 9 is a block diagram of an example computing system in communication with a fabrication system that can be used to process customized input and fabricate customized face sheets and masks in accordance with the present disclosure.

FIG. 10 is a flowchart illustrating an example process that may be used in fabricating a customizable face sheet and corresponding mask.

The same reference numbers or other reference designators are used in the drawings to designate the same or similar (functionally and/or structurally) features. The figures are not necessarily drawn to scale.

### DETAILED DESCRIPTION

Applying cosmetics or medications to the face can be a time-consuming endeavor, yet this activity is something many people do daily, if not multiple times a day. While engaged in applying certain cosmetics or medications to the face, which can at times require one's full attention, it may be challenging to simultaneously perform other activities effectively. There is a need for a solution in which a person can consistently apply customized cosmetics and other skincare to selected surface areas of that person's face in a manner that would enable the person to simultaneously perform other tasks.

Certain examples described herein provide a customizable cosmetic delivery system that can be specially configured to ensure even and precise application of various cosmetics targeting specific facial areas. The cosmetic delivery system can include a face sheet. The face sheet can be customized, for example, to transfer selected cosmetics specific to a wearer's preferred skin tones, colors, brands, facial placement, etc. The cosmetic delivery system can also include a mask configured to facilitate proper placement of the face sheet on a wearer's face. The mask can be customized, for example, to conform comfortably to the contours of a wearer's face, ensuring an optimal and customized fit. Some implementations can integrate seamlessly into a frequently-used cosmetic routine. A wearer can enjoy a revitalizing and nourishing treatment as customizable cosmetics are transferred from the facial mask to the skin, even while the wearer performs other wholly unrelated tasks, thereby offering a near effortless, spa-like experience in the comfort of one's home.

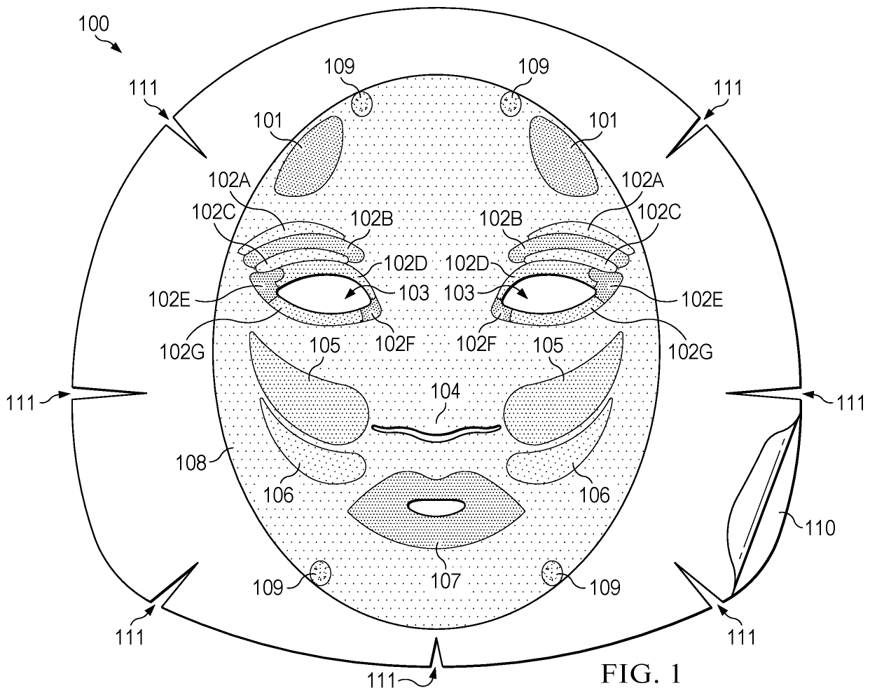

FIG. 1 is an example of a face sheet 100 having a facial shape and an inner surface 110 removably attachable to facial skin. The inner surface 110 has formed thereon a plurality of biocompatible cosmetics 101-108. In some examples, each cosmetic 101-108 is transferable to facial skin responsive to compressing the face sheet 100 against a human face. The face sheet 100 can be composed of a soft, hypoallergenic material, for example, which can be configured to gently adhere to facial skin and create a barrier that promotes the absorption of cosmetics 101-108.

In some examples, face sheet 100 may be substantially planar in shape, yet face sheet 100 may be configured to form a substantially continuous attachment to the three-dimensional contours of a wearer's face. For example, to facilitate such a continuous attachment, face sheet 100 may be sufficiently flexible to be bent in half without plastic deformation thereof and may include one or more cutouts 111 positioned at various locations around a periphery of face sheet 110. Another cutout 204 positioned proximate the position of a noise may further allow the face sheet to conform to a wearer's face while providing an opening to facilitate breathing. Eye cutouts 203 positioned proximate the position of the eyes may enhance comfort of a wearer and enable the wearer to have an unobstructed view while the face sheet 100 is applied to the wearer's face. In some examples, face sheet 100 may include multiple divided or divisible portions, which may further facilitate conforming the face sheet 100 to respective portions of the wearer's face. For example, face sheet 100 may include one or more upper portions that is separate from, or that is attached to and is separatable from (e.g., through a curve or line of perforations formed therethrough) one or more lower portions.

The cosmetics 101-108 (on inner surface 110 of face sheet 100) include any suitable biocompatible product that can be applied to a human face to immediately enhance or alter a person's appearance. For example, cosmetics 101-108 may collectively include foundation, concealer, powder, blush, contour, highlight, eyeshadow, eyeliner, lipstick, and so forth. Foundation can be used, for example, to create an even skin tone. Concealers can be used, for example, to help cover specific blemishes or imperfections. Various powders can be applied, for example, to set the makeup and to reduce shine. Various blushes can be used, for example, to add specific color proximate to the cheeks. Contouring products can be used, for example, in deepening of the skin to create a shadow and bring out what is next to it. Highlighting products can be applied, for example, to facial areas to be accentuated and thus can be used in cooperation with contouring products. Eyeshadow and eyeliner can be used, for example, to enhance features around the eyes. Lipstick or lip gloss can be used, for example, to provide color and texture to the lips.

In some examples, cosmetics 101-108 may be customized according to the needs of a given wearer. The customization may include, for example, enabling selection or confirmation of which specific cosmetics 101-108 are applied (e.g., in terms of product type, brand, and color) and where they are to be applied (e.g., in terms of the specific locational positioning, total surface area, and shape of the cosmetics 101-108 applied to the inner surface 110 of face sheet). In the example shown in FIG. 1, for example, one or more highlighting products 101 are applied relative to proximate left and right peripheries of the forehead, one or more blush products 105 are applied in customized patterns relative to proximate positioning of the left and right cheeks, one or more contouring products 106 are applied in customized patterns beneath respective patterns of blush products 105, one or more lip products 107 are applied relative to the proximate the position of the lips, and one or more foundation products 108 are applied. One or more highlighting products 102A, contouring products 102B, crease/fold products 102C, eyelid products 102D, outer-eye-corner products 102E, inner-eye-corner products 102F, and lower-lash-line products 102G are applied at respective positions relative to the eyes, as shown in FIG. 1.

In some examples, the foundation products 108 can be applied to portions of face sheet 100 corresponding to the entire surface area of a human face. In some examples, the foundation products 108 can be applied to the face sheet 100 in an inverse pattern that corresponds to portions of the face not covered by one or more of products 101-107.

The face sheet 100 may include one or more medications on inner surface 110, in addition to cosmetics 101-108. The medication may be integrated within one or more of the cosmetics 101-108 or may occupy a respective surface area of face sheet 100 that does not have any cosmetics 101-108 applied thereto. The medication may include sunscreen, moisturizer, or any other suitable skin medication. In some instances, the medication may be configured to penetrate into facial skin without altering a person's appearance.

In some examples, face sheet 100 may have a plurality of distinct trays or wells formed on inner surface 110. Each tray or well may have a recessed portion configured to contain a certain volume of a respective cosmetic 101-108. The recessed portion may have a respective maximum depth to accommodate a desired volume of cosmetic per a given surface area, where different trays or wells may have different maximum depths as needed. In some examples, a tray or well may include a bubble protruding from inner surface 110 to accommodate an additional volume of cosmetic per surface area.

A thin plastic sheet may be removably attached to inner surface 110 and configured to provide a protective and peelable covering that reduces the risk of cosmetics 101-108 from drying out and from being smudged or unintentionally separated from face sheet 100 (e.g., while in transit). The plastic sheet may have holes formed therein, or may have separatable portions thereof, consistent with the pattern of biocompatible adhesive dots 109, which in some instances may facilitate peeling the remainder of plastic sheet from inner surface 110. The plastic sheet may also have holes therethrough (e.g., pinholes) that are positioned in a manner that facilitates the transfer of underlying cosmetics to facial skin, including even while the plastic sheet remains fixed in place on the face sheet 100.

The inner surface 110 also has thereon (e.g., proximate to a periphery thereof) a plurality of biocompatible adhesive dots 109. Adhesive dots 109 can be configured, including in terms of their material properties, respective surface area, shape, and placement on inner surface 110, to removably attach face sheet 100 to facial skin and to remain attached to face sheet 100 responsive to detachment of the face sheet 100 from facial skin. Any suitable biocompatible adhesive may be used for each adhesive dot 109 including, for example spirit gum, liquid latex, certain acrylic adhesives, certain silicone adhesives, acrylate (designed for medical/surgical use), etc.

As noted above, the face sheet 100 shown in FIG. 1 may be customized to accommodate the desired effect for a particular wearer. In some instances, the specific cosmetics 101-108 that are selectively applied (e.g., in terms of product type, brand, and color) and where they are to be applied (e.g., in terms of the specific locational positioning, total surface area, and shape of the cosmetics 101-108 applied to the inner surface 110 of face sheet) may depend, at least in part, on the shape of the wearer's face.

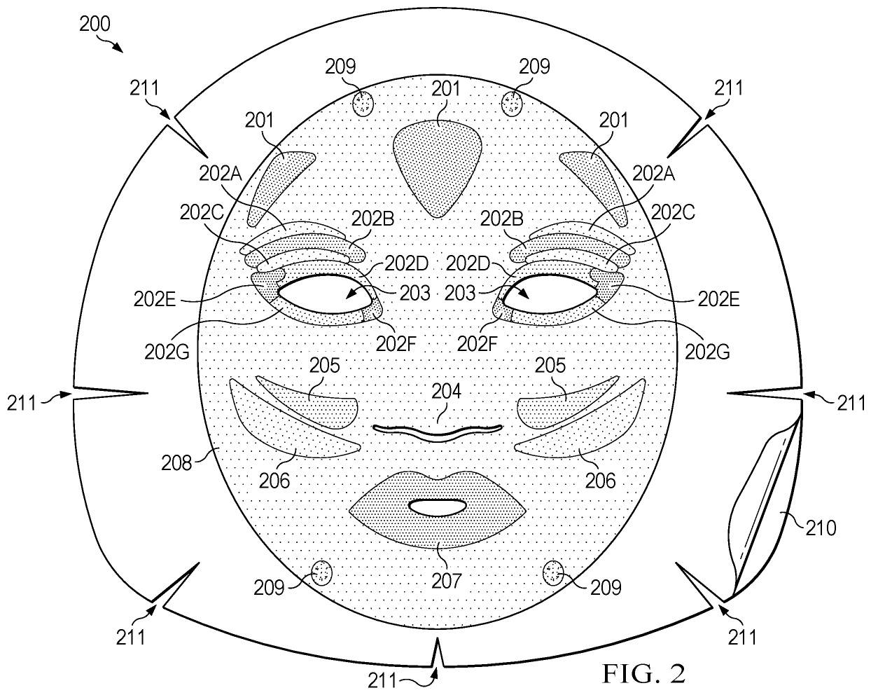

FIG. 2 is a top-down view of an example face sheet 200 according to a different customization. Face sheet 200 may be customized, for example, for a rounder facial shape (e.g., relative to that of FIG. 1). Face sheet 200 has an inner surface 210 removably attachable to facial skin and a plurality of biocompatible cosmetics 201-208 formed thereon. Each cosmetic 201-208 is configured to be transferable to facial skin responsive to compressing the face sheet 200 against a human face. For example, one or more highlighting products 201 are applied relative to proximate left, right, and center portions of the forehead, one or more blush products 205 are applied in customized patterns relative to proximate positioning of the left and right cheeks, one or more contouring products 206 are applied in customized patterns beneath respective patterns of blush products 205, one or more lip products 207 are applied relative to the proximate the position of the lips, and one or more foundation products 208 are applied in an inverse pattern that approximately covers the remainder of the face. In addition, one or more highlighting products 202A, contouring products 202B, crease/fold products 202C, eyelid products 202D, outer-eye-corner products 202E, inner-eye-corner products 202F, and lower-lash-line products 202G are applied at respective positions relative to the eyes, as shown in FIG. 2. The inner surface 210 also has thereon (e.g., proximate to a periphery thereof) a plurality of biocompatible adhesive dots 209 similar in structure and function to adhesive dots 109 of FIG. 1.

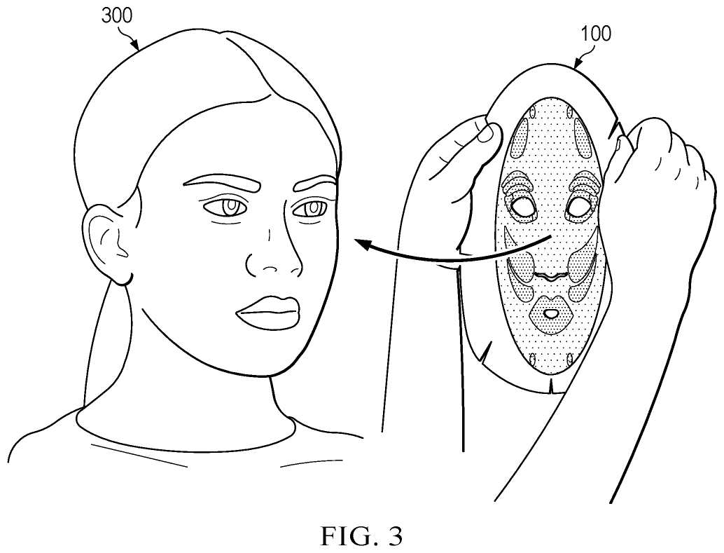

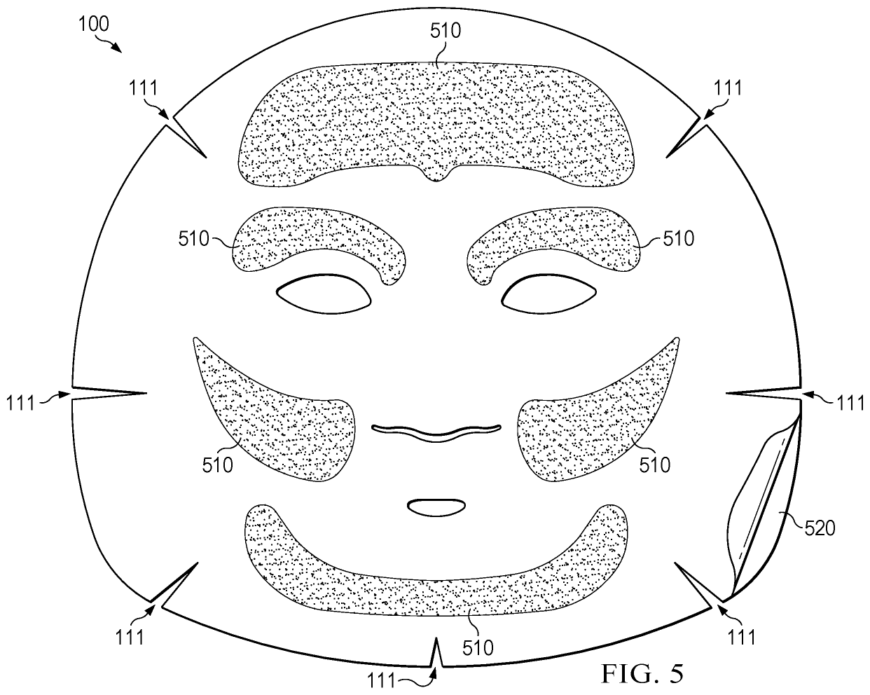

FIG. 3 is a side-view of a wearer 300 preparing to manually apply face sheet 100 to her face, with inner surface 110 (having cosmetics 101-108 disposed thereon) shown as facing the wearer's face. Certain individuals may find it challenging to place a face sheet (e.g., face sheets 100/200) consistently and accurately at the intended location on the face, in order to accurately cause the transfer of cosmetics (e.g., 101-108/201-208) to facial skin at the intended locations. As explained further with reference to FIGS. 5-8, a hard-mask positioning system may be used, which, among other technical advantages, can facilitate the accurate and consistent positioning of face sheet 100/200 on a wearer's face, such that cosmetics 101-108 can be accurately transferred to facial skin at the intended locations.

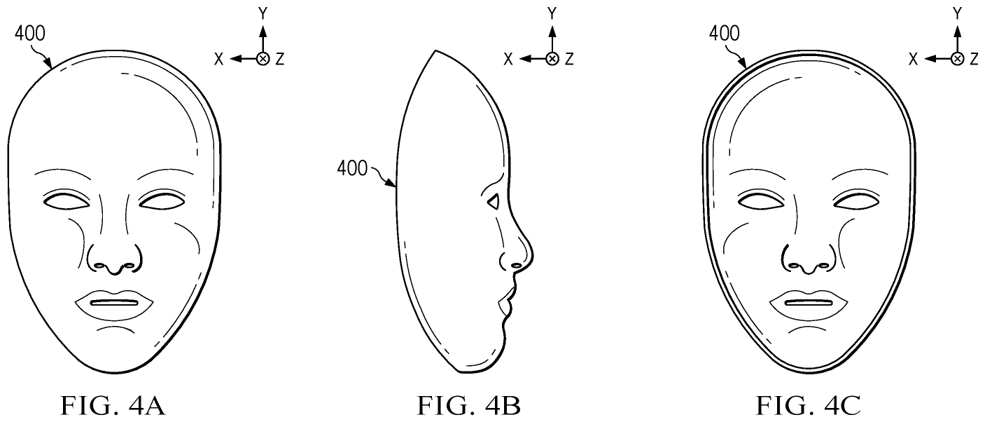

FIGS. 4A-4C show various perspective views of a portion of an example mask 400, in which FIG. 4A is a front view, FIG. 4B is a side-view, and FIG. 4C is a back view. The illustrated x-y-z axis illustrates the relative viewing angle of each FIG. 4A-4C. In some examples, mask 400 is customized in three mutually-orthogonal dimensions to fit the contours of a specific wearer's face. As shown in FIGS. 4A-4C, mask 400 may include cutouts corresponding to respective positioning of a wearer's eyes, nose, and mouth. Mask 400 may be fabricated mostly from plastic using 3D printing, for example. However, any suitable material and customizable manufacturing may be used. In certain embodiments, mask 400 may include a heating mechanism (e.g., powered by a battery or wired source) that is configured to distribute heat along the inner skin-facing surface of mask 400. In some instances, the distribution of heat along an inner surface of mask 400 may facilitate the transfer of cosmetics 101-108 to the facial skin of a wearer. As explained further with reference to FIGS. 5-8, mask 400 may be part of a positioning system that assists a wearer to accurately position face sheet 100/200 on the wearer's face, thereby facilitating the accurate transfer of cosmetics 101-108/201-208 to the intended facial locations.

FIG. 5 is a top-down view of the outer surface 520 of the face sheet 100 of FIG. 1, which is opposite inner surface 110. Outer surface 520 can have adhesive 510 formed thereon and positioned to create sufficient adhesion of face sheet 100 to mask 400 when applied thereto, such that when mask 400 is manually lifted off of a wearer's face, face sheet 100 remains adhered to mask 400 and thus also cleanly lifts off the wearer's face. Any suitable adhesive 510 may be used in any suitable pattern. For example, adhesive 510 may be applied in a pattern that at least encompasses surface arears corresponding to adhesive dots 109 applied to the inner surface 110. Unlike dots 109, however, adhesive 510 need not be biocompatible as it is applied to back surface 520 and hence not intended to come in contact with facial skin.

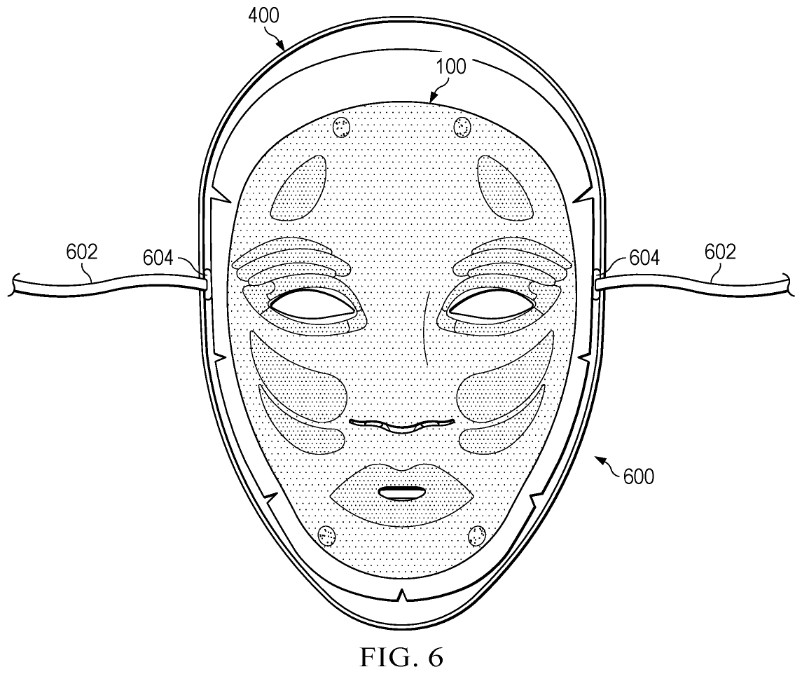

FIG. 6 is a perspective view of a positioning system 600 including face sheet 100 positioned within mask 400, in which outer surface 520 of face sheet 100 opposes an inner surface of mask 400 and adhesive 510 holds mask 400 in its proper place relative to mask 400 without slippage. In this preparatory configuration, cosmetics 101-108 are exposed and outward facing on inner surface 110 of face sheet 100. The illustrated configuration may assist a wearer in accurately transferring the customized cosmetics 101-108 to facial skin, as the three-dimensional hard mask 400 can provide a tactile indication of facial features and thereby assist in aligning hard mask 400 relative to the wearer's corresponding facial features. Mask 400 may include one or more straps 602 attached to mask 400 at attachment points 604 and configured to hold mask 400 in place on a wearer's face. Once positioning system 600 is in contact with a wearer's face, mask 400 may provide a relatively rigid surface that more evenly distributes compressive force manually-applied to an outer surface of mask 400. The manually-applied compressive force may cause the customized cosmetics 101-108 to facial skin at the intended locations on the wearer's face.

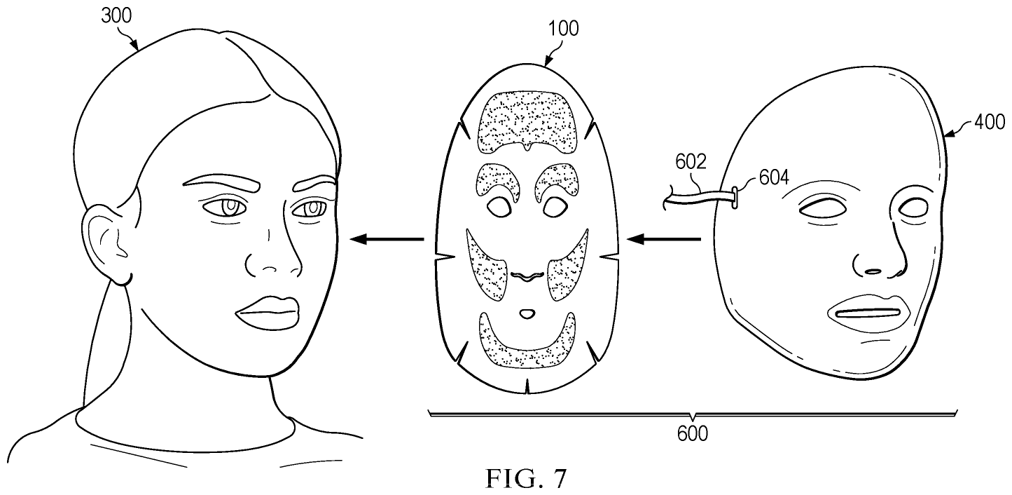

FIG. 7 shows an exploded side-view illustrating the orientation of positioning system 600 relative to a wearer's face, with inner surface 110 (having cosmetics 101-108 disposed thereon) shown as facing the wearer's face and outer surface 510 shown as facing an inner surface of mask 400.

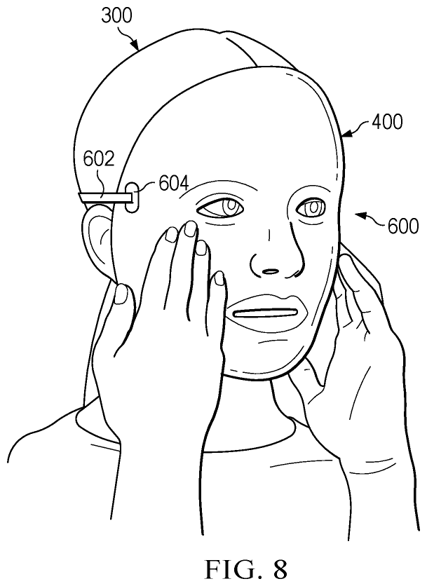

FIG. 8 is a side-view of positioning system 600, in which a wearer has manipulated positioning system 600 against her face and is in the process of manually compressing positioning system 600 against her face, thereby initiating transfer of cosmetics 101-108 to facial skin at their intended locations. The wearer may opt to drop her hands at some point during the cosmetic transfer process, in which case straps 602 will assist in keeping positioning system 600 in place. As noted previously, positioning system 600 may be equipped with a heating mechanism that can be activated once positioning system 600 is in place and the transfer of cosmetics 101-108 has been initiated. In some examples, the application of heat may facilitate the release and more fulsome transfer of cosmetics 101-108 to facial skin.

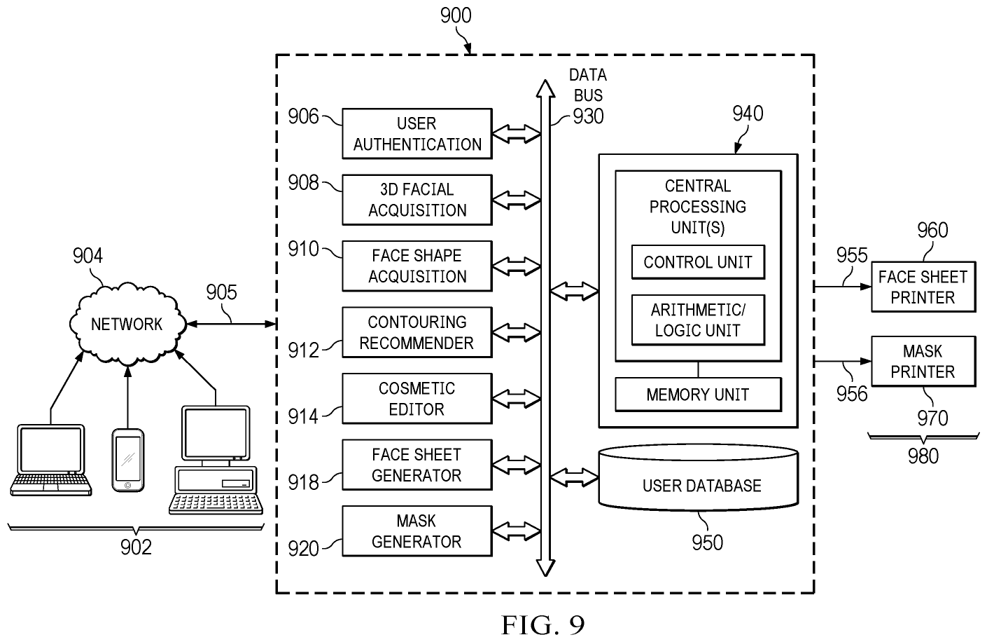

FIG. 9 is a block diagram of an example computing system 900 in communication with a fabrication system 980 that can be used to process customized input and fabricate a customized face sheet (e.g., face sheets 100/200) and masks (e.g., mask 400) in accordance with the present disclosure. In this example, computing system 900 is configured to receive customized input from one or more devices 902 (e.g., a mobile phone, laptop computer, desktop computer, etc.) via a network (e.g., the Internet). The customized input can include, for example, one or more images of a person's face (e.g., from various angles), which computing system 900 can use to generate a three-dimensional digital model of the person's face. The customized input can also include, for example, selections pertaining to cosmetics to be applied to a face mask (e.g., in terms of type, color, brand, or intended transfer location on a person's face).

Computing system 900 includes various specialized computing components 906-950 in communication with one another (e.g., via data base 930) and operable to process various input 905 (e.g., received via network 904 from input devices 902) and to produce corresponding output 955 specially formatted, for example, for use in fabricating a customized face sheet (e.g., face sheet 100/200) and a corresponding customized face mask (e.g., face mask 400).

Processor 940 refers to one or more computer processors configured to process instructions of special-purpose computer programs, so as to realize a variety of functions of computing system 900. Processors 940 can include one or more Central Processing Unit(s) (CPU) in communication with one or more Control Units and Arithmetic/Logic Unit, for example. A Memory Unit may be used to assist in processing functions. The Memory Unit refers to one or more memory devices capable of storing digital information. Such a memory device may be a read-only memory (ROM), random access memory (RAM), volatile memory, non-volatile memory, static memory, dynamic memory, flash memory, cache memory, frame memory, or any device that stores digital information.

A user database 950 can be used to store information pertaining to a user. For example, user database 950 can store for each the corresponding login credentials, payment systems, use history, preferences, and user selections applied in generating a face sheet and corresponding mask setup.

User authentication 906 contains executable computing logic for authenticating users (e.g., based on user information stored in user database 950).

3D facial acquisition 908 contains executable computing logic for rendering three-dimensional digital representations of a person's face (e.g., based on two-dimensional still images, or a series of images in a video stream, of that person's face).

Face shape acquisition 910 contains executable computing logic for categorizing a three-dimensional digital representation of a user's face in terms of shape. For example, face shape acquisition may use algorithms to determine whether a three-dimensional digital representation of a user's face most closely approximates an oval shape, a round shape, a long shape, a square shape, a rectangular or oblong shape, a triangle shape, a diamond shape, an inverted triangle shape, a heart shape. However, any suitable collection of face-shape categories may be used.

Contouring recommender 912 contains executable computing logic configured to output (e.g., for display to a user) certain cosmetic configurations (e.g., in terms of type of cosmetic, color, facial placement, brand, etc.). In some examples, the recommended cosmetic configurations are based on an output of face shape acquisition 910. The output of contouring recommender 912 may thus be specially-configured and customized for a particular user based on input received from that user. In some examples, the output of contouring recommender 912 may be based on a cosmetic profile of another individual, such as a beautician, celebrity, influencer, who has who has granted permission to share the same. In some examples, the output of contouring recommender 912 may be based on a cosmetic profile generated at least in part using artificial intelligence algorithms.

Cosmetic editor 914 contains executable computing logic configured to enable a user to selectively edit cosmetics to be applied to a face sheet (e.g., in terms of type of cosmetic(s), or their respective color, facial placement, brand, etc.) or certain aspects of a corresponding face mask (e.g., the overall shape, the size or positioning of eye, nose, or mouth openings, etc.). As a starting point, cosmetic editor 914 may be configured to send displayable data to a user representative of the output of contouring recommender 912 or a stored cosmetic profile for a user (e.g., as stored in user database 950).

Face sheet generator 918 contains executable computing logic configured to generate digital output 955 useable in generating a face sheet responsive to one or more of the other computing components 906-918 of system 900.

Mask generator 920 contains executable computing logic configured to generate digital output 956 useable in generating a mask responsive to one or more of the other computing components 906-918 of system 900.

Fabrication system 980 includes a face sheet printer subsystem 960 and a mask printer subsystem 970. Face sheet printer subsystem 960 applies customized cosmetics to an inner surface of a face sheet based on output 955. The application of customized cosmetics to the face sheet may involve, for example, a professional hand-injecting at least some of the customized cosmetics or applying touchups to customized cosmetics previously applied to a face sheet by a printing machine. Mask printer subsystem 970 produces a three-dimensional digital mask (e.g., mask 400) based on output 956. For example, mask printer subsystem 970 may include a 3D printer configured to produce a customized hard-plastic and reusable mask 400 based on output 956.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Herein, “and” is both joint and several, unless expressly indicated otherwise or indicated otherwise by context. Thus, “A and B” means “A and B, jointly or severally,” unless expressly indicated otherwise or indicated otherwise by context. To aid the Patent Office, and any readers of any patent issued on this application, in interpreting the claims appended hereto, there is no intention that any of the appended claims invoke 35 U.S.C. § 112(f) as it exists on the date of filing hereof unless the words “means for” or “step for” are explicitly used in the claim language.

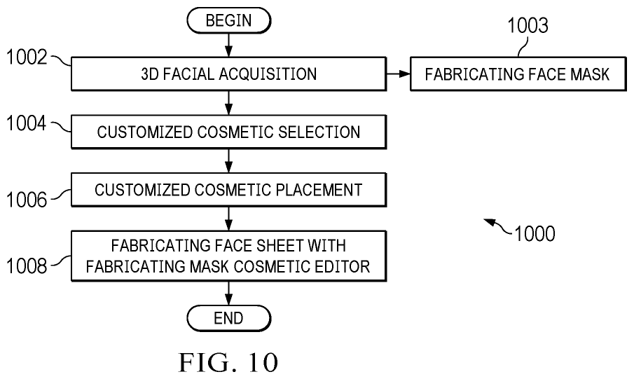

FIG. 10 is a flowchart 1000 illustrating an example process that may be used in fabricating a customizable face sheet and corresponding mask. Process 1000 includes 3D facial acquisition (1002). For example, based on image data received from a user (e.g., from one or more devices 902 and via network 904), 3D facial acquisition 908 can execute computing logic for rendering a three-dimensional digital representation of a person's face (e.g., based on two-dimensional still images, or a series of images in a video stream, of that uses' face) (1002).

Process 1000 optionally includes fabricating a face mask (1003). For example, a face mask (e.g., mask 400) can be fabricated using the three-dimensional digital representation of the person's face generated as output in executing step 1002.

Process 1000 further includes customized cosmetic selection (1004). For example, cosmetic editor 914 can execute computing logic enabling a user (e.g., through interaction with a graphical user interface) to selectively chose one or more cosmetics to be applied to a face sheet (e.g., in terms of type of cosmetic(s), or their respective color, brand, etc.) and a respective facial placement area for each chosen cosmetic (1004).

Process 1000 further includes physically fabricating a face sheet (1008) (e.g., based on the results of steps 1002-1006). For example, the face-sheet fabrication (1008) may be based on first data corresponding to a three-dimensional image of a person's face, second data corresponding to a customized selection of a plurality of cosmetics, and third data corresponding to a customized selection of a respective facial surface area for each of the plurality of cosmetics. The face-sheet fabrication (1008) may involve face sheet generator 918 executing computing logic configured to generate digital output 955 useable in generating a face sheet responsive to one or more of the other computing components 906-918 of system 900. The digital output 955 may be specially formatted, for example, for use in machine-fabricating at least a portion of a customized face sheet (e.g., face sheet 100/200) having customized cosmetics applied to an inner surface 110/210 thereof (1008). The face-sheet fabrication (1008) may include removably attaching a first cosmetic of the plurality of user-selected cosmetics to a first inner surface area of a face sheet, the first inner surface area corresponding to a customized selection of the respective facial surface area, the first cosmetic being configured to attach to human skin responsive to pressure applied to an outer surface of the face sheet.

Herein, a device that is described herein as including certain components may instead be adapted to be coupled to those components to form the described device. A device described herein is reconfigurable to include additional or different components to provide functionality at least partially similar to functionality available prior to the component replacement. A device that is “configured to” perform a task or function may be configured (e.g., programmed and/or hardwired) at a time of manufacturing by a manufacturer to perform the function and/or may be configurable (or reconfigurable) by a user after manufacturing to perform the function and/or other additional or alternative functions. The configuring may be through firmware and/or software programming of the device, through a construction and/or layout of hardware components and interconnections of the device, or a combination thereof. In addition, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. The recitation “based on” means “based at least in part on.” Therefore, if X is based on Y, then X may be a function of Y and any number of other factors.

This disclosure encompasses all changes, substitutions, variations, alterations, and modifications to the examples herein that a person having ordinary skill in the art would comprehend. Similarly, where appropriate, the appended claims encompass all changes, substitutions, variations, alterations, and modifications to the examples herein that a person having ordinary skill in the art would comprehend.

What is claimed is:

1. An apparatus comprising:
a face sheet having a facial shape and an inner surface removably attachable to facial skin;
a plurality of biocompatible cosmetics on the inner surface, each cosmetic being transferable to facial skin responsive to compressing the face sheet against a human face; and
a biocompatible adhesive on the inner surface, the biocompatible adhesive configured to removably attach the face sheet to facial skin and to remain attached to the face sheet responsive to detachment of the face sheet from facial skin.

2. The apparatus of claim 1, further comprising:
a mask configured to receive the face sheet therein, the mask having three-dimensional contours customized to fit a wearer's face; and
a mask adhesive configured to adhere the face sheet to the mask and to maintain adhesion between the face sheet and the mask responsive to detachment of the face sheet and mask from facial skin.

3. The apparatus of claim 1, wherein the biocompatible cosmetics are water soluble.

4. The apparatus of claim 1, wherein the inner surface of the face sheet has a surface area of at least 20 square inches.

5. The apparatus of claim 1, wherein, the plurality of cosmetics include a first cosmetic at a first location on the inner surface of the face sheet and a second cosmetic at a second location on the inner surface of the face sheet, the first and second cosmetics and the first and second locations corresponding to customized user choices.

6. The apparatus of claim 5, wherein the first and second locations are selected from the group consisting of: a superior orbital rim of an eye, an eyelid, a cheek, a lip, a forehead, under eye, a nose, a chin, and an entire face.

7. The apparatus of claim 1, wherein the face sheet is planar.

8. The apparatus of claim 1, wherein the face sheet is sufficiently flexible to bend in half.

9. The apparatus of claim 1, wherein the face sheet has a plurality of openings therethrough, each opening corresponding to a respective position of an eye, nose, or mouth.

10. The apparatus of claim 1, wherein the face sheet is made of biomaterials or polymers.

11. The apparatus of claim 1, wherein the plurality of cosmetics include a foundation product.

12. The apparatus of claim 1, wherein the plurality of cosmetics are selected from the group consisting of: eyeshadow, concealer, foundation, blush, and contour.

13. The apparatus of claim 1, further comprising medication removably attached to the inner surface of the face sheet and configured to penetrate into human skin.

14. The apparatus of claim 13, wherein the medication is either sunscreen or moisturizer.

15. A method comprising:
accessing first data corresponding to a three-dimensional image of a human face;
accessing second data corresponding to a customized selection of a plurality of cosmetics;
accessing third data corresponding to a customized selection of a respective facial surface area for each of the plurality of cosmetics; and
based on the first data, the second data, and the third data, removably attaching a first cosmetic of the plurality of user-selected cosmetics to a first inner surface area of a face sheet, the first inner surface area corresponding to the customized selection of the respective facial surface area, the first cosmetic being configured to attach to human skin responsive to pressure applied to an outer surface of the face sheet.

16. The method of claim 15, further comprising applying skin-friendly adhesive on the inner surface of the face sheet, the skin-friendly adhesive configured to removably adhere the face sheet to human skin.

17. The method of claim 15, further comprising:
based on the first data, fabricating a mask customized in three mutually-orthogonal dimensions to fit the human face; and
applying a mask adhesive on an outer surface of the face sheet, the mask adhesive configured to adhere the face sheet to the mask and to maintain adhesion between the face sheet and the mask responsive to a lift-off force manually applied to the mask, the lift-off force being sufficient to simultaneously lift the mask and the face sheet adhered thereto off the human face.

18. The method of claim 15, wherein the respective facial feature is selected from the group consisting of: a superior orbital rim of an eye, an eyelid, a cheek, a lip, a forehead, under eye, a nose, a chin, and an entire face.

19. The method of claim 15, wherein a first cosmetic of the plurality of cosmetics is a foundation and the respective facial surface area for the first cosmetic corresponds to at least half of the three-dimensional image of the human face.

20. An apparatus comprising:
a planar face sheet having an inner surface and an outer surface;
a plurality of biocompatible cosmetics on the inner surface;
a mask customized in three mutually-orthogonal dimensions to fit a human face; and
adhesive on the outer surface of the planar face sheet, the adhesive configured to removably attach the face sheet to the mask and to hold the face sheet in place within the mask;
wherein each biocompatible cosmetic is configured to transfer to facial skin, and thereby alter an appearance of facial skin, responsive to a force manually applied to an outer surface of the mask.
